(19) United States
(12) Patent Application Publication
(10) Pub. No.: US 2002/0012646 A1
(43) Pub. Date: Jan. 31, 2002

(54) Shampoo compositions with cationic polymers

(76) Inventors: Douglas Allan Royce, Aurora, IN (US); Robert Lee Wells, Cincinnati, OH (US); Eric Scott Johnson, Hamilton, OH (US); Jacob Daniel Taylor, Bensenville, IL (US); Kiichiro Nakamura, Higashinada-ku (JP); Jian-Zhong Yang, Higashinada-ku (JP)

Correspondence Address: Linda M. Sivik, The Procter & Gamble Company, Sharon Woods Technical Center, 11511 Reed Hartman Highway, Cincinnati, OH, 45241, US

(21) Appl. No.: 09/853,227

(22) Filed: May 11, 2001

Related U.S. Application Data
(63) Continuation-in-part of application No. 09/662,084, filed on Sep. 14, 2000.
Application No. 09/662,084 is a continuation-in-part of application No. 08/852,166, filed on May 6, 1997.

(51) Int. Cl. A61K 7/11; A61K 7/075

(52) U.S. Cl. 424/70.11; 424/70.13

(57) ABSTRACT

Disclosed are hair conditioning shampoo compositions comprising: a) from about 5% to about 50%, by weight, of a surfactant component selected from the group consisting of anionic surfactants, amphoteric surfactants, or a combination of anionic and amphoteric or zwitterionic surfactants where the amphoteric surfactants are anionic or zwitterionic at the pH of the composition; b) from about 0.01% to about 5%, by weight, of a water soluble, organic, cationic polymer hair conditioning agent having a cationic charge density of from about 0.1 meq/gram to about 1.2 meq/gram and wherein said water soluble, organic, cationic polymer hair conditioning agent has a molecular weight greater than 600,000; and c) an aqueous carrier.

### CROSS REFERENCE TO RELATED APPLICATION

This application is a continuation-in-part of prior application Ser. No. 09/662,084 filed on Sep. 14, 2000 which in turn is a continuation-in-part Ser. No. 08/852,166 filed on May 6, 1997.

### FIELD OF THE INVENTION

This invention relates to shampoo compositions containing hair conditioning ingredients.

### BACKGROUND OF THE INVENTION

Human hair becomes soiled due to its contact with the surrounding atmosphere and, to a greater extent, from sebum secreted by the head. The build-up of the sebum causes the hair to have a dirty feel and an unattractive appearance. The soiling of the hair necessitates it being shampooed with frequent regularity.

Shampooing the hair cleans by removing excess soil and sebum. However, the shampooing process has disadvantages in that the hair is left in a wet, tangled and generally unmanageable state. Shampooing can also result in the hair becoming dry or “frizzy”, and a loss of luster, due to removal of natural oils or other hair moisturizing materials. After shampooing, the hair can also suffer from a loss of “softness” perceived by the user upon drying. The hair can also suffer from increased levels of static upon drying after shampooing. This can interfere with combing and can result in “fly-away” hair. A variety of approaches have been developed to alleviate the after-shampoo problems. These range from the inclusion of hair conditioning aids in shampoos to post-shampoo application of hair conditioners, i.e., hair rinses. Hair rinses are generally liquid in nature and must be applied in a separate step following the shampooing, left on the hair for a length of time, and rinsed with fresh water. This, of course, is time consuming and is not as convenient as shampoos containing both cleaning and hair conditioning ingredients.

While a wide variety of shampoos have been disclosed which contain conditioning aids, they have not been totally satisfactory for a variety of reasons. Cationic conditioning agents are highly desirable for use in hair conditioning due to their abilities to control static, improve wet detangling, and provide a silky wet hair feel to the user. One problem which has been encountered in shampoos relates to compatibility problems between good cleaning anionic surfactants and the many conventional cationic agents which historically have been used as conditioning agents. Efforts have been made to minimize adverse interaction through the use of alternate surfactants and improved cationic conditioning agents. Cationic surfactants which provide good overall conditioning in hair rinse products, in general, tend to complex with anionic cleaning surfactants and provide poor conditioning in a shampoo context. In particular, the use of soluble cationic surfactants that form soluble ionic complexes do not deposit well on the hair. Soluble cationic surfactants that form insoluble ionic complexes deposit on the hair but do not provide good hair conditioning benefits, and tend to cause the hair to have a dirty, coated feel. The use of insoluble cationic surfactants, e.g., tricetyl methyl ammonium chloride, can provide excellent anti-static benefits but do not otherwise provide good overall conditioning. Many cationic polymers tend to build up on the hair to result in an undesirable, “unclean” coated feel. Cationic polymers therefore, conventionally, are preferably used at limited levels to minimize this problem. This, however, can limit the overall conditioning benefits that are obtained. It has been found in the art, for example in U.S. Pat. No. 5,186,928, Birtwistle, Feb. 16, 1993, that higher charge density polymers are superior as deposition aids for small particle dispersed agents.

Cationic conditioning agents commonly do not provide optimal overall conditioning benefits, particularly in the area of “softness”, especially when delivered as an ingredient in a shampoo composition. Materials which can provide increased softness are nonionic silicones. Silicones in shampoo compositions have been disclosed in a number of different publications. Such publications include U.S. Pat. No. 2,826,551, Geen, issued Mar. 11, 1958; U.S. Pat. No. 3,964,500, Drakoff, issued Jun. 22, 1976; U.S. Pat. No. 4,364,837, Pader, issued Dec. 21, 1982; and British Patent 849,433, Woolston, issued Sep. 28, 1960. While these patents disclose silicone containing compositions, they do not provide a totally satisfactory product in that it difficult to maintain the silicone well dispersed and suspended in the product. Stable, insoluble silicone-containing hair conditioning shampoo compositions have been described in U.S. Pat. No. 4,741,855, Grote and Russell, issued May 3, 1988 and U.S. Pat. No. 4,788,066, Bolich and Williams, issued Nov. 29, 1988.

Improved conditioning shampoos are provided U.S. Pat. No. 5,573,709 issued on Nov. 12, 1996. Japanese Patent Application, Laid Open No. 56-72095, Jun. 16, 1981, Hirota et al. (Kao Soap Corp.) also discloses shampoo containing cationic polymer and silicone conditioning agents. Still other patent publications relating to shampoos with cationic agents and silicone include EPO Application Publication 0 413 417, published Feb. 20, 1991, Hartnett et al.

Another approach to providing hair conditioning benefits to shampoo compositions has been to use materials which are oily to the touch. These materials provide improved luster and shine to the hair. Oily materials have also been combined with cationic materials in the shampoo formulations as disclosed in Japanese Patent Application Showa 53-35902, laid open Oct. 6, 1979 (Showa 54-129135), N. Uchino (Lion Yushi Co.) and Japanese Patent Application 62 [1987]-327266, filed Dec. 25, 1987, published Jul. 4, 1989, laid open No. HEI 1[1987]-168612, Horie et al.

In spite of these attempts to provide optimal combinations of cleaning ability and hair conditioning, it remains desirable to provide further improved hair conditioning shampoo compositions. For instance, it remains desirable to improve overall conditioning, and especially shine and luster, wet and dry combing, and dry hair feel, of hair treated with shampoo containing silicone and cationic material.

One attempt to do this is disclosed in EPO Patent Publication No. 0 413 416, published Feb. 20, 1991, Robbins et al., which discloses shampoo containing aminosilicone, anionic surfactant, cationic surfactant, and a hydrocarbon component. These types of formulations would normally be expected to result in either excessive buildup of aminosilicone on the hair, and consequently greasy hair feel and loss of fullness, or a relatively limited degree of improvement due to intentional use of very low levels of aminosilicone to avoid such adverse effects. The cationic surfactants would have limited ability to condition the hair due to interaction with the anionic surfactant.

Other patent documents which disclose shampoo compositions and a variety of conditioning agents are EPO Patent Application Publication No. 0 413 417, published Feb. 20, 1991, U.S. Pat. No. 3,964,500, Drakoff, issued Jun. 22, 1976 and U.S. Pat. No. 5,085,857 (Reid et al.).

In spite of all these approaches and attempts to provide optimum combinations of shampoos and hair conditioners, it remains desirable to provide still improved conditioning shampoos.

### SUMMARY OF THE INVENTION

The present invention is directed to hair conditioning shampoo compositions comprising: (a) from about 5% to about 50%, by weight, of a surfactant component selected from the group consisting of anionic surfactants, amphoteric surfactants, or a combination of anionic and amphoteric or zwitterionic surfactants where the amphoteric surfactants are anionic or zwitterionic at the pH of the composition; (b) from about 0.01% to about 5%, by weight, of water soluble, organic, cationic polymer hair conditioning agent having a cationic charge density of from about 0.1 meq/gram to about 1.2 meq/gram, the cationic polymer having a molecular weight greater than 600,000; and (c) an aqueous carrier.

The invention, including preferred embodiments thereof, is described in further detail in the Detailed Description of the Invention, which follows.

### DETAILED DESCRIPTION OF THE INVENTION

While the specification concludes with claims which particularly point out and distinctly claim the invention, it is believed the present invention will be better understood from the following description.

The present invention addresses the need for improved conditioning shampoos, by providing hair conditioning shampoo compositions having from about 5% to about 50%, by weight, of a surfactant component selected from the group consisting of anionic surfactants, amphoteric surfactants, or a combination of anionic and amphoteric or zwitterionic surfactants where the amphoteric surfactants are anionic or zwitterionic at the pH of the composition; from about 0.01% to about 5%, by weight, of water soluble, organic, cationic polymer hair conditioning agent having a cationic charge density of from about 0.1 meq/gram to about 1.2 meq/gram, the cationic polymer having a molecular weight greater than 600,000; and an aqueous carrier.

As discussed above, it has been previously known that higher charge density polymers are superior as deposition aids for small particle dispersed agents. However, we have surprisingly found that low charge density cationic polymers, although they are less efficient as deposition aids, are in fact better than the higher charge density cationic polymers for providing wet conditioning benefits.

Without being bound by theory, it is believed that the wet conditioning benefits are a result of the formation of a complex coacervate either in the full formula or during the wash or rinse step during shampoo use. This wet coacervate deposits on hair and delivers the wet conditioning benefit. Although the coacervate formation is caused by charge attraction of the anionic micelles and cationic polymers, it has surprisingly been found that the amount of this coacervate actually increases as the charge density of the cationic polymer decreases. Thus, the lower charge density cationic polymer will yield higher levels of coacervate and therefore higher wet conditioning.

We have also surprisingly discovered that the cationic polymers that form larger amounts of coacervate also form coacervates that contain lower levels of non-volatiles. Such coacervates with less non-volatiles provide the benefit of leaving the hair with a cleaner feel after drying and result in less weigh down to the hair. Therefore, not only do the low charge density cationic polymers provide the benefit of improved wet conditioning, but they provide this benefit without leaving as much residue on the hair as would be expected if higher levels of less efficient polymers were used.

Consequently, it has now been found that improved overall conditioning can be found by combining anionic surfactant in a shampoo with a soluble cationic organic polymer hair conditioning agent of low charge density and high molecular weight. These compositions can provide improved conditioning while reducing the level of undesirable side effects that can result from excessive deposition of conditioning agent in prior known conditioning systems. As discussed previously, a conditioning agent system with high charge density cationic polymers can result in build up on the hair over repeated usages and to loss of fullness of the hair. Too much cationic conditioning agent results in a coated, dirty feel of the hair. Now it has been found that the components of the present invention can provide improved overall conditioning while minimizing the adverse effects of conditioning agent build-up that otherwise can be incurred upon increasing the levels of individual components in prior known conditioning systems.

The present invention is directed to providing shampoo compositions which can provide excellent cleaning performance, improved lathering, and improved levels of conditioning while minimizing any adverse side effects associated with build-up due to the use of excess conditioning agent.

The present invention is further directed to a method for cleaning and conditioning the hair which can provide excellent cleaning in combination with improved conditioning, while minimizing adverse side effects associated with excess build-up of conditioning agent on the hair.

These and other features, aspects, and advantages of the present invention will become evident to those skilled in the art from a reading of the present disclosure with the appended claims.

The essential components and properties of the compositions of the present invention are described below. A nonexclusive description of various optional and preferred components useful in embodiments of the present invention is also described below.

The shampoo compositions of the present invention can comprise, consist of, or consist essentially of the essential elements and limitations of the invention described herein, as well as any of the additional or optional ingredients, components, or limitations described herein.

All percentages, parts and ratios are based upon the total weight of the shampoo compositions of the present invention, unless otherwise specified. All such weights as they pertain to listed ingredients are based on the active level and, therefore, do not include carriers or by-products that may be included in commercially available materials, unless otherwise specified.

Herein, “soluble” refers to any material that is sufficiently soluble in water to form a substantially clear solution to the naked eye at a concentration of 0.1% by weight of the material in water at 25° C., unless otherwise specifically indicated. Conversely, the term “insoluble” refers to all other materials that are therefore not sufficiently soluble in water to form a substantially clear solution to the naked eye at a concentration of 0.1% by weight of the other material in water at 25° C., unless otherwise specifically indicated.

Herein, “liquid” refers to any visibly (by the naked eye) flowable fluid under ambient conditions (about 1 atmosphere of pressure at about 25° C.)

All cited references are incorporated herein by reference in their entireties. Citation of any reference is not an admission regarding any determination as to its availability as prior art to the claimed invention.

### I. Anionic Detersive Surfactant Component

The hair conditioning shampoo compositions of the present invention contain an anionic surfactant component, which can comprise one or more anionic detersive surfactants, amphoteric surfactants or the combination of anionic and zwitterionic surfactant, wherein the amphoteric detersive surfactants are anionic or zwitterionic at the pH of the shampoo, to provide cleaning performance to the composition.

The anionic surfactant component will generally be present at a level from about 5% to about 50%, preferably from about 8% to about 30%, more preferably from about 10% to about 25%, even more preferably from about 12% to about 20%, by weight of the composition.

Anionic detersive detergents useful herein include alkyl and alkyl ether sulfates. These materials have the respective formulae ROSO₃M and RO(C₂H₄O)ₓSO₃M, wherein R is alkyl or alkenyl of from about 8 to about 24 carbon atoms, x is 1 to 10, and M is a water-soluble cation such as ammonium, alkanolamines, such as triethanolamine, monovalent metals, such as sodium and potassium and polyvalent metal cations, such as magnesium, and calcium. The cation M, of the anionic detersive surfactant should be chosen such that the detersive surfactant component is water soluble. Solubility will depend upon particular anionic detersive surfactants and cations chosen.

The alkyl ether sulfates are typically made as condensation products of ethylene oxide and monohydric alcohols having from about 8 to about 24 carbon atoms. Preferably, R has from about 8 to about 18 carbon atoms, more preferably from about 10 to about 16 carbon atoms, more preferably still from about 12 to about 14 carbon atoms in both the alkyl and alkyl ether sulfates. The alcohols can be derived from fats, e.g., coconut oil, palm kernal or tallow, or can be synthetic. Lauryl alcohol and straight chain alcohols derived from coconut oil or palm kernal are preferred herein. Such alcohols are reacted with about 1 to about 10, and especially about 3, molar proportions of ethylene oxide and the resulting mixture of molecular species having, for example, an average of 3 moles of ethylene oxide per mole of alcohol, is sulfated and neutralized.

Specific examples of alkyl ether sulfates which may be used in the present invention are sodium and ammonium salts of coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, the mixture having an average alkyl chain length of from about 10 to about 16 carbon atoms and an average degree of ethoxylation of from about 1 to about 4 moles of ethylene oxide. Such a mixture also comprises from about 0 to about 20% by weight C₁₂₋₁₃ compounds; from about 0 to about 20% by weight of C1₁₇₋₁₈₋₁₉ compounds; from about 3 to about 30% by weight of compounds having a degree of ethoxylation of 0; from about 45 to about 90% by weight of compounds having a degree of ethoxylation of from about 1 to about 4; from about 10 to about 25% by weight of compounds having a degree of ethoxylation of from about 4 to about 8; and from about 0.1 to about 15% by weight of compounds having a degree of ethoxylation greater than about 8.

Another suitable class of anionic detersive surfactants are the water-soluble salts of the organic, sulfuric acid reaction products of the general formula:

R₁—SO₃—M

wherein R₁ is chosen from the group consisting of a straight or branched chain, saturated aliphatic hydrocarbon radical having from about 8 to about 24, preferably about 10 to about 18, carbon atoms; and M is a cation. Examples are the salts of an organic sulfuric acid reaction product of a hydrocarbon of the methane series, including iso-, neo-, ineso-, and n-paraffins, having about 8 to about 24 carbon atoms, preferably about 12 to about 18 carbon atoms and a sulfonating agent, e.g., SO3, H₂SO₄, oleum, obtained according to known sulfonation methods, including bleaching and hydrolysis. Preferred are alkali metal and ammonium sulfonated C₁₀₋₁₈ n-parrafins.

Additional examples of synthetic anionic detersive surfactants which come within the terms of the present invention are the reaction products of fatty acids esterified with isethionic acid and neutralized with sodium hydroxide where, for example, the fatty acids are derived from coconut oil or palm kernal; sodium or potassium salts of fatty acid amides of methyl tauride in which the fatty acids, for example, are derived from coconut oil or palm kernal. Other similar synthetic anionic detersive surfactants of this variety are set forth in U.S. Pat. Nos. 2,486,921; 2,486,922; and 2,396,278.

Still other synthetic anionic detersive surfactants useful in the present invention are in the class designated as succinamates. This class includes such surface active agents as disodium N-octadecylsulfosuccinamate; disodium lauryl sulfosuccinate; diammonium lauryl sulfosuccinate; tetrasodium N-(1,2-dicarboxyethyl)-N-octadecylsulfosuccinamate; diamyl ester of sodium sulfosuccinic acid; dihexyl ester of sodium sulfosuccinic acid; dioctyl esters of sodium sulfosuccinic acid.

Other useful suitable anionic detersive surfactants include olefin sulfonates having about 10 to about 24 carbon atoms. Herein, “olefin sulfonates means compounds which can be produced by the sulfonation of alpha-olefins by means of uncomplexed sulfur trioxide, followed by neutralization of the acid reaction mixture in conditions such that any sulfones which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydrocarbons, etc., when used in the liquid form, or by air, nitrogen, gaseous SO₂, etc., when used in the gaseous form.

The alpha-olefins from which the olefin sulfonates are derived are mono-olefins having about 10 to about 24 carbon atoms, preferably about 12 to about 16 carbon atoms. Preferably, they are straight chain olefins.

In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process.

A specific alpha-olefin sulfonate mixture of the above type is described more fully in the U.S. Pat. No. 3,332,880, Pflaumer and Kessler, issued Jul. 25, 1967.

Another class of anionic detersive surfactants useful in the present invention are the beta-alkyloxy alkane sulfonates. These compounds have the following formula:

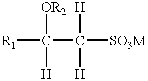

where R₁ is a straight chain alkyl group having from about 6 to about 20 carbon atoms, R₂ is a lower alkyl group having from about 1 (preferred) to about 3 carbon atoms, and M is a water-soluble cation as hereinbefore described.

Many additional synthetic anionic surfactants useful in the present invention are described in McCutcheon's, Emulsifiers and Detergents, 1989 Annual, published by M. C. Publishing Co. Also U.S. Pat. No. 3,929,678, Laughlin et al., issued Dec. 30, 1975, discloses many other anionic as well as other useful surfactant types.

Preferred anionic detersive surfactants for use in the present shampoo compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, and sodium dodecyl benzene sulfonate and combinations thereof.

### A. Amphoteric and Zwitterionic Surfactants

Suitable amphoteric surfactant components for use in the shampoo compositions herein include those which are known for use in shampoo compositions or other personal care cleansing composition, and which contain a group that is anionic or zwitterionic at the pH of the shampoo composition. Examples of amphoteric surfactants suitable for use in the shampoo composition herein are described in U.S. Pat. No. 5,104,646 (Bolich Jr. et al.), U.S. Pat. No. 5,106,609 (Bolich Jr. et al.).

Examples of amphoteric detersive surfactants which can be used in the compositions of the present invention are those which are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, cocoamphoacetate, cocoamphodiacetate, lauroamphoacetate, lauroamphodiacetate, sodium lauryl sarcosinate, sodium lauroamphoacetate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products sold under the trade name “MIRANOL”™ and described in U.S. Pat. No. 2,528,378.

Zwitterionic detersive surfactants are exemplified by those which can be broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. A general formula for these compounds is:

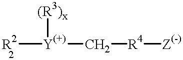

wherein R contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Other zwitterionics such as betaines can also useful in the present invention. Examples of betaines useful herein include the high alkyl betaines, such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alpha-carboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in this invention.

### B. Optional Detersive Surfactants

In addition to the anionic detersive surfactant component, the compositions of the present invention can optionally contain other detersive surfactants. These include nonionic surfactants. Optional detersive surfactants, when used, are typically present at levels of from about 0.5% to about 20%, more typically from about 1% to about 10%, although higher or lower levels can be used. The total amount of detersive surfactant in compositions containing optional detersive surfactants in addition to the anionic surfactant will generally be from about 5.5% to about 50%, preferably from about 8% to about 30%, more preferably from about 10% to about 25%. Cationic detersive surfactants can also be used, but are generally less preferred because they can adversely interact with the anionic detersive surfactant. Cationic detersive surfactants, if used, are preferably used at levels no greater than about 5%. Cationic surfactants, if used, are more typically conditioning agents which can optionally be included in the compositions hereof.

Nonionic detersive surfactants which can be used include those broadly defined as compounds produced by the condensation of alkylene oxide groups (hydrophilic in nature) with an organic hydrophobic compound, which may be aliphatic or alkyl aromatic in nature. Examples of preferred classes of nonionic detersive surfactants are:

1. The polyethylene oxide condensates of alkyl phenols, e.g., the condensation products of alkyl phenols having an alkyl group containing from about 6 to about 20 carbon atoms in either a straight chain or branched chain configuration, with ethylene oxide, the said ethylene oxide being present in amounts equal to from about 10 to about 60 moles of ethylene oxide per mole of alkyl phenol.

2. Those derived from the condensation of ethylene oxide with the product resulting from the reaction of propylene oxide and ethylene diamine products.

3. The condensation product of aliphatic alcohols having from about 8 to about 18 carbon atoms, in either straight chain or branched chain configuration, with ethylene oxide, e.g., a coconut alcohol ethylene oxide condensate having from about 10 to about 30 moles of ethylene oxide per mole of coconut alcohol, the coconut alcohol fraction having from about 10 to about 14 carbon atoms.

4. Long chain tertiary amine oxides corresponding to the following general formula:

R₁R₂R₃N—>O

wherein R₁ contains an alkyl, alkenyl or monohydroxy alkyl radical of from about 8 to about 18 carbon atoms, from 0 to about 10 ethylene oxide moieties, and from 0 to about 1 glyceryl moiety, and R₂ and R₃ contain from about 1 to about 3 carbon atoms and from 0 to about 1 hydroxy group, e.g., methyl, ethyl, propyl, hydroxyethyl, or hydroxypropyl radicals. The arrow in the formula is a conventional representation of a semipolar bond.

5. Long chain tertiary phosphine oxides corresponding to the following general formula:

RR′R″P—>O

wherein R contains an alkyl, alkenyl or monohydroxyalkyl radical ranging from about 8 to about 18 carbon atoms in chain length, from 0 to about 10 ethylene oxide moieties and from 0 to about 1 glyceryl moiety and R′ and R″ are each alkyl or monohydroxyalkyl groups containing from about 1 to about 3 carbon atoms.

6. Long chain dialkyl sulfoxides containing one short chain alkyl or hydroxy alkyl radical of from about 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to about 1 glyceryl moiety.

7. Alkyl polysaccharide (APS) surfactants such as the alkyl polyglycosides. Such surfactants are described in U.S. Pat. No. 4,565,647, Llenado, issued Jan. 21, 1986, incorporated herein by reference, which discloses APS surfactants having a hydrophobic group with about 6 to about 30 carbon atoms and polysaccharide (e.g., polyglycoside) as the hydrophilic group. Optionally, there can be a polyalkylene-oxide group joining the hydrophobic and hydrophilic moieties. The alkyl group (i.e., the hydrophobic moiety) can be saturated or unsaturated, branched or unbranched, and unsubstituted or substituted (e.g., with hydroxy or cyclic rings).

8. Polyethylene glycol (PEG) glyceryl fatty esters, such as those of the formula R(O)OCH₂CH(OH)CH₂(OCH₂CH₂)ₙOH wherein n is from about 5 to about 200, preferably from about 20 to about 100, and R is an aliphatic hydrocarbyl having from about 8 to about 20 carbon atoms.

Preferred shampoos of the present invention contain combinations of anionic surfactants with zwitterionic surfactants and/or amphoteric surfactants. Preferred shampoos contain from about 0% to about 16% of alkyl sulfates, from 0% to about 16% of ethoxylated alkyl sulfates, and from about 0% to about 10% of optional detersive surfactants selected from the nonionic, amphoteric, and zwitterionic detersive surfactants, with at least 5% of either alkyl sulfate, ethoxylated alkyl sulfate, or a mixture thereof, and a total surfactant level of from about 10% to about 25%.

### II. Cationic Polymer Hair Conditioning Agent

The shampoo compositions of the present invention further comprise a water soluble, cationic organic polymer hair conditioning. The polymeric cationic hair conditioning agent hereof will generally be present at levels of from about 0.01% to about 5%, preferably from about 0.05% to about 4%, more preferably from about 0.1% to about 3%, by weight, of the shampoo composition.

The cationic organic polymers useful in the hair conditioning agent hereof are organic polymers that can provide conditioning benefits to hair and that are soluble in the shampoo composition or form liquid coacervates in the shampoo composition. Any cationic polymers which can provide these benefits can be used. Herein, “polymer” shall include materials whether made by polymerization of one type of monomer or made by two (i.e., copolymers) or more types of monomers or natural occurring polymers.

The cationic charge density is in a range from about 0.1 meq/gram to about 1.2 meq/gram, preferably in a range from about 0.3 to about 0.8, more preferably in a range from about 0.5 to about 0.7, wherein the cationic polymer has a molecular weight greater than 600,000, preferably in a range from about 800,000 to about 2 million, more preferably in range from about 1 million to about 1.5 million, more preferably still in a range from about 1.25 million to about 1.35 million. Preferably, the cationic polymer will have a molecular weight less than 5 million.

Cationic charge density of the cationic polymer can be determined according to the Kjeldahl Method. Those skilled in the art will recognize that the charge density of amino-containing polymers may vary depending upon pH and the isoelectric point of the amino groups. The charge density should be within the above limits at the pH of intended use, which will in general be from about pH 3 to about pH 9, most generally from about pH 4 to about pH 8.

Any anionic counterions can be utilized for the cationic polymers so long as the water solubility criteria is met. Suitable counterions include halides (e.g., Cl, Br, I, or F, preferably Cl, Br, or I), sulfate, and methylsulfate. Others can also be used, as this list is not exclusive.

The cationic nitrogen-containing moiety will be present generally as a substituent, on a fraction of the total monomer units of the cationic hair conditioning polymers. Thus, the cationic polymer can comprise copolymers, terpolymers, etc. of quaternary ammonium or cationic amine-substituted monomer units and other non-cationic units referred to herein as spacer monomer units. Such polymers are known in the art, and a variety can be found in the CTFA Cosmetic Ingredient Dictionary, 3rd edition, edited by Estrin, Crosley, and Haynes, (The Cosmetic, Toiletry, and Fragrance Association, Inc., Washington, D.C., 1982).

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. The alkyl and dialkyl substituted monomers preferably have C₁-C₇ alkyl groups, more preferably C₁-C₃ alkyl groups. Other suitable spacer monomers include vinyl esters, vinyl alcohol (made by hydrolysis of polyvinyl acetate), maleic anhydride, propylene glycol, and ethylene glycol.

The cationic amines can be primary, secondary, or tertiary amines, depending upon the particular species and the pH of the shampoo. In general, secondary and tertiary amines, especially tertiary amines, are preferred.

Amine-substituted vinyl monomers can be polymerized in the amine form, and then optionally can be converted to ammonium by a quaternization reaction. Amines can also be similarly quaternized subsequent to formation of the polymer. For example, tertiary amine functionalities can be quaternized by reaction with a salt of the formula R′X wherein R′ is a short chain alkyl, preferably a C₁-C₇ alkyl, more preferably a C₁-C₃ alkyl, and X is an anion which forms a water soluble salt with the quaternized ammonium.

Suitable cationic amino and quaternary ammonium monomers include, for example, vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts. The alkyl portions of these monomers are preferably lower alkyls such as the C₁-C₃ alkyls, more preferably C₁ and C₂ alkyls.

Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide, wherein the alkyl groups are preferably C₁-C₇ hydrocarbyls, more preferably C₁-C₃, alkyls.

The cationic polymers hereof can comprise mixtures of monomer units derived from amine- and/or quaternary ammonium-substituted monomer and/or compatible spacer monomers.

Suitable cationic hair conditioning polymers include, for example: copolymers of 1-vinyl-2-pyrrolidone and 1-vinyl-3-methylimidazolium salt (e.g., chloride salt); copolymers of 1-vinyl-2-pyrrolidone and dimethylaminoethyl methacrylate (referred to in the industry by CTFA as Polyquaternium-11) such as those commercially available from Gaf Corporation (Wayne, N.J., USA) under the GAFQUAT tradename (e.g., GAFQUAT 755N); cationic diallyl quaternary ammonium-containing polymers, including, for example, copolymers of acrylamide and dimethyldiallylammonium chloride, and mineral acid salts of amino-alkyl esters of homo- and co-polymers of unsaturated carboxylic acids having from 3 to 5 carbon atoms, as described in U.S. Pat. No. 4,009,256.

Other cationic polymers that can be used include polysaccharide polymers, such as cationic cellulose derivatives, cationic guar and cationic starch derivatives.

Cationic polysaccharide polymer materials suitable for use herein include those of the formula:

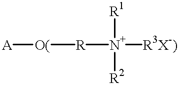

wherein: A is an anhydroglucose residual group, such as a starch or cellulose anhydroglucose residual, R is an alkylene oxyalkylene, polyoxyalkylene, or hydroxyalkylene group, or combination thereof; R¹, R₂, and R³ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R² and R³) preferably being about 20 or less, and X is an anionic counterion, as previously described.

Cationic cellulose is available from Amerchol Corp. (Edison, N.J., USA) in their Polymer LR series of polymers, as salts of hydroxyethyl cellulose reacted with trimethyl ammonium substituted epoxide, referred to in the industry (CTFA) as Polyquaternium 10. A preferred Polyquaternium-10 cationic cellulosic polymer for the present invention is available from Amerchol Corp. under the tradename Polymer LR-30M. LR-30M has a cationic charge density of 0.7 meq/g and a molecular weight of 1,250,000.

Another type of cationic cellulose includes the polymeric quaternary ammonium salts of hydroxyethyl cellulose reacted with lauryl dimethyl ammonium-substituted opoxide, referred to in the industry (CTFA) as Polyquaternium 24.

Other cationic polymers that can be used include cationic guar gum derivatives, such as guar hydroxypropyltrimonium chloride (commercially available from Celanese Corp. in their JaguarR series). Other materials include quaternary nitrogen-containing cellulose ethers (e.g., as described in U.S. Pat. No. 3,962,418 and copolymers of etherified cellulose and starch, as described in U.S. Pat. No. 3,958,581.

As discussed above, the cationic polymer hereof is water soluble. This does not mean, however, that it must be soluble in the shampoo composition. Preferably however, the cationic polymer is either soluble in the shampoo composition, or in a complex coacervate phase in the shampoo composition formed by the cationic polymer and anionic material. Complex coacervates of the cationic polymer can be formed with anionic surfactants or with anionic polymers that can optionally be added to the compositions hereof (e.g., sodium polystyrene sulfonate).

Coacervate formation is dependent upon a variety of criteria such as molecular weight, concentration, and ratio of interacting ionic materials, ionic strength (including modification of ionic strength, for example, by addition of salts), charge density of the cationic and anionic species, pH, and temperature. Coacervate systems and the effect of these parameters has previously been studied. See, for example, J. Caelles, et al., “Anionic and Cationic Compounds in Mixed Systems”, Cosmetics & Toiletries, Vol. 106, April 1991, pp 49-54, C. J. van Oss, “Coacervation, Complex-Coacervation and Flocculation”, J. Dispersion Science and Technology, Vol. 9 (5,6), 1988-89, pp 561-573, and D. J. Burgess, “Practical Analysis of Complex Coacervate Systems”, J. of Colloid and Interface Science, Vol. 140, No. 1, November 1990, pp 227-238.

Complex coacervates are believed to readily deposit on the hair. Thus, in general, it is preferred that the cationic polymer exist in the shampoo as a coacervate phase or form a coacervate phase upon dilution. If not already a coacervate in the shampoo, the cationic polymer will preferably exist in a complex coacervate form in the shampoo upon dilution with water to a water:shampoo composition weight ratio of about 20:1, more preferably at about 10:1, even more preferably at about 8:1.

Techniques for analysis of formation of complex coacervates are known in the art. For example, microscopic analyses of the shampoo compositions, at any chosen stage of dilution, can be utilized to identify whether a coacervate phase has formed. Such coacervate phase will be identifiable as an additional emulsified phase in the composition. The use of dyes can aid in distinguishing the coacervate phase from other insoluble phases dispersed in the composition.

Exemplary complex coacervate shampoo compositions are shown in the examples. Many other cationic polymers, depending upon the other parameters of the shampoo composition, can also form coacervates, as will be understood by those skilled in the art.

It has been found that for compositions containing cationic polymer conditioning agents having cationic charge density and molecular weight within the above range can provide enhanced conditioning performance and coacervate formation.

### III. Insoluble Hair Conditioning Agent

The shampoo compositions of the present invention may further comprise an insoluble hair conditioning agent at concentrations effective to provide hair conditioning benefits. Such concentrations generally range from about 0.005% to about 5%, preferably from about 0.05% to about 4%, more preferably from about 0.1% to about 3.5%, most preferably from about 0.2% to about 3%, by weight of the shampoo compositions. The insoluble hair conditioning particles useful in the present invention have a particle size range less than or equal to 50 microns, preferably less than or equal to 35 micron, most preferably less than or equal to 28 microns. Useful conditioning agents include silicone, petrolatum, and hair conditioning oily liquid such as hydrocarbon oils, fatty esters, synthetic esters and mixtures.

### A. Silicone Hair Conditioning Agent

The shampoo compositions of the present invention may further comprise a non-volatile, nonionic or cationic silicone hair conditioning agent and mixtures thereof, which are insoluble in the shampoo compositions hereof. The silicone hair conditioning agent is intermixed in the shampoo composition so as to be in the form of dispersed, insoluble particles, or droplets. The silicone hair conditioning agent comprises a nonvolatile, insoluble, silicone fluid and optionally comprises a silicone gum which is insoluble in the shampoo composition as a whole but is soluble in the silicone fluid. The silicone hair conditioning agent can also comprise other ingredients, such as a silicone resin to enhance deposition efficiency.

Herein, “nonvolatile” refers to silicone material with little or no significant vapor pressure under ambient conditions, as is understood by those in the art. Boiling point under one atmosphere (atm) will preferably be at least about 250° C., more preferably at least about 275° C., most preferably at least about 300° C. Vapor pressure is preferably about 0.2 mm HG at 25° C. or less, preferably about 0.1 mm HG at 25° C. or less.

The silicone hair conditioning agent may comprise low levels of volatile silicone components; however, such volatile silicones will preferably exceed no more than about 0.5%, by weight, of the shampoo composition. Typically, if volatile silicones are present, it will be incidental to their use as a solvent or carrier for commercially available forms of other ingredients, such as silicone gums and resins.

The silicone hair conditioning agent for use herein will preferably have viscosity of from about 1,000 to about 2,000,000 centistokes at 25° C., more preferably from about 10,000 to about 1,800,000, even more preferably from about 100,000 to about 1,500,000. The viscosity can be measured by means of a glass capillary viscometer as set forth in Dow Coming Corporate Test Method CTM0004, Jul. 20, 1970.

The silicone hair conditioning agent will be used in the shampoo compositions hereof at levels of from about 0.05% to about 10% by weight of the composition, preferably from about 0.1% to about 10%, more preferably from about 0.5% to about 8%, most preferably from about 0.5% to about 5%.

Suitable insoluble, nonvolatile silicone fluids include polyalkyl siloxanes, polyaryl siloxanes, polyalkylaryl siloxanes, polyether siloxane copolymers, and mixtures thereof. Other insoluble, nonvolatile silicone fluids having hair conditioning properties can also be used. Herein, “silicone fluid” shall mean flowable silicone materials having a viscosity of less than 1,000,000 centistokes at 25° C. Generally, the viscosity of the fluid will be between about 5 and 1,000,000 centistokes at 25° C., preferably between about 10 and about 100,000.

Silicone fluids hereof also include polyalkyl or polyaryl siloxanes with the following structure:

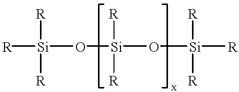

where R is aliphatic, preferably alkyl or alkenyl, or aryl, R can be substituted or unsubstituted, and x is an integer from 1 to about 8,000. Suitable unsubstituted R groups include alkoxy, aryloxy, alkaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

The aliphatic or aryl groups substituted on the siloxane chain may have any structure as long as the resulting silicones remain fluid at room temperature, are hydrophobic, are neither irritating, toxic nor otherwise harmful when applied to the hair, are compatible with the other components of the shampoo compositions, are chemically stable under normal use and storage conditions, are insoluble in the shampoo compositions, and are capable of being deposited on and, of conditioning, the hair.

The two R groups on the silicon atom of each monomeric silicone unit may represent the same group or different groups. Preferably, the two R groups represent the same group.

Preferred alkyl and alkenyl substituents are C₁-C₅ alkyls and alkenyls, more preferably from C₁-C₄, most preferably from C₁-C₂. The aliphatic portions of other alkyl-, alkenyl-, or alkynyl-containing groups (such as alkoxy, alkaryl, and alkamino) can be straight or branched chains and preferably have from one to five carbon atoms, more preferably from one to four carbon atoms, even more preferably from one to three carbon atoms, most preferably from one to two carbon atoms. As discussed above, the R substituents hereof can also contain amino functionalities, e.g. alkamino groups, which can be primary, secondary or tertiary amines or quaternary ammonium. These include mono-, di- and tri- alkylamino and alkoxyamino groups wherein the aliphatic portion chain length is preferably as described above. The R substituents can also be substituted with other groups, such as halogens (e.g. chloride, fluoride, and bromide), halogenated aliphatic or aryl groups, and hydroxy (e.g. hydroxy substituted aliphatic groups). Suitable halogenated R groups could include, for example, tri-halogenated (preferably fluoro) alkyl groups such as —R¹—C(F)₃, wherein R¹ is C₁-C₃ alkyl. Examples of such polysiloxanes include polymethyl-3,3,3 trifluoropropylsiloxane.

Suitable R groups include methyl, ethyl, propyl, phenyl, methylphenyl and phenylmethyl. The preferred silicones are polydimethyl siloxane, polydiethylsiloxane, and polymethylphenylsiloxane. Polydimethylsiloxane is especially preferred. Other suitable R groups include methyl, methoxy, ethoxy, propoxy, and aryloxy. The three R groups on the end caps of the silicone may also represent the same or different groups.

The nonvolatile polyalkylsiloxane fluids that may be used include, for example, polydimethylsiloxanes. These siloxanes are available, for example, from the General Electric Company in their ViscasilR and SF 96 series, and from Dow Coming in their Dow Coming 200 series.

The polyalkylaryl siloxane fluids that may be used, also include, for example, polymethylphenylsiloxanes. These siloxanes are available, for example, from the General Electric Company as SF 1075 methyl phenyl fluid or from Dow Coming as 556 Cosmetic Grade Fluid.

The polyether siloxane copolymers that may be used include, for example, a polypropylene oxide modified polydimethylsiloxane (e.g., Dow Coming DC-1248) although ethylene oxide or mixtures of ethylene oxide and propylene oxide may also be used. The ethylene oxide and polypropylene oxide level must be sufficiently low to prevent solubility in water and the composition hereof.

References disclosing suitable silicone fluids include U.S. Pat. No. 2,826,551, Geen; U.S. Pat. No. 3,964,500, Drakoff, issued Jun. 22, 1976; U.S. Pat. No. 4,364,837, Pader; and British Patent 849,433, Woolston. Also incorporated herein by reference is Silicon Compounds distributed by Petrarch Systems, Inc., 1984. This reference provides an extensive (though not exclusive) listing of suitable silicone fluids.

Another silicone material that can be especially useful in the silicone conditioning agents is insoluble silicone gum. The term “silicone gum”, as used herein, means polyorganosiloxane materials having a viscosity at 25° C. of greater than or equal to 1,000,000 centistokes. Silicone gums are described by Petrarch and others including U.S. Pat. No. 4,152,416, Spitzer et al., issued May 1, 1979 and Noll, Walter, Chemistry and Technology of Silicones, New York: Academic Press 1968. Also describing silicone gums are General Electric Silicone Rubber Product Data Sheets SE 30, SE 33, SE 54 and SE 76. All of these described references are incorporated herein by reference. The “silicone gums” will typically have a mass molecular weight in excess of about 200,000, generally between about 200,000 and about 1,000,000. Specific examples include polydimethylsiloxane, (polydimethylsiloxane) (methylvinylsiloxane) copolymer, poly(dimethylsiloxane) (diphenyl siloxane)(methylvinylsiloxane) copolymer and mixtures thereof.

The silicone hair conditioning agent may comprise a mixture of a polydimethylsiloxane gum, having a viscosity greater than about 1,000,000 centistokes and polydimethylsiloxane fluid having a viscosity of from about 10 centistokes to about 100,000 centistokes, wherein the ratio of gum to fluid is from about 30:70 to about 70:30, preferably from about 40:60 to about 60:40.

Another optional ingredient that can be included in the silicone conditioning agent is silicone resin. Silicone resins are highly crosslinked polymeric siloxane systems. The crosslinking is introduced through the incorporation of trifunctional and tetrafunctional silanes with monofunctional or difunctional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units (and hence, a sufficient level of crosslinking) such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinyl-chlorosilanes, and tetrachlorosilane, with the methyl-substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art.

Background material on silicones including sections discussing silicone fluids, gums, and resins, as well as manufacture of silicones, can be found in Encyclopedia of Polymer Science and Engineering, Volume 15, Second Edition, pp 204-308, John Wiley & Sons, Inc., 1989.

Silicone materials and silicone resins in particular, can conveniently be identified according to a shorthand nomenclature system well known to those skilled in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}%; and Q denotes the quadri- or tetra-functional unit SiO₂. Primes of the unit symbols, e.g., M′, D′, T′, and Q′ denote substituents other than methyl, and must be specifically defined for each occurrence. Typical alternate substituents include groups such as vinyl, phenyls, amines, hydroxyls, etc. The molar ratios of the various units, either in terms of subscripts to the symbols indicating the total number of each type of unit in the silicone (or an average thereof) or as specifically indicated ratios in combination with molecular weight complete the description of the silicone material under the MDTQ system. Higher relative molar amounts of T, Q, T′ and/or Q′ to D, D′, M and/or or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MQ and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000.

The weight ratio of the nonvolatile silicone fluid component to the silicone resin component, when used, is from about 4:1 to about 400:1, preferably this ratio is from about 9:1 to about 200:1, more preferably from about 19:1 to about 100:1, particularly when the silicone fluid component is a polydimethylsiloxane fluid or a mixture of polydimethylsiloxane fluid and polydimethylsiloxane gum as described above.

### B. Synthetic Esters

The shampoo composition of the present invention may comprise select synthetic esters at concentrations ranging from about 0.01% to about 1.0%, preferably from about 0.05% to about 0.5%, more preferably from about 0.08% to about 0.3%, by weight of the shampoo composition. These select esters provide improved wet hair feel when used in combination with the essential components of the shampoo composition herein, and in particular when used in combination with the cationic hair conditioning polymer described hereinbefore.

The synthetic esters for use in the shampoo composition are water insoluble and have a viscosity of from about 1 to about 300 centipoise, preferably from about 1 to about 150 centipoise, more preferably from about 2 to about 50 centipoise. The synthetic esters conform to either of the following Formulas I or II.

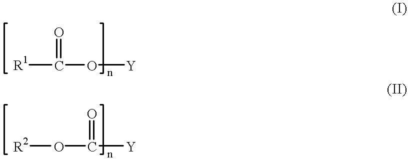

wherein R¹ is an alkyl, alkenyl, hydroxyalkyl or hydroxyalkenyl group, having from 7 to 9 carbon atoms, preferably a saturated alkyl group, more preferably a saturated, linear, alkyl group; n is a positive integer having a value of from 2 to 4, preferably 3; R² is an alkyl, alkenyl, hydroxyalkyl or hydroxyalkenyl group, having from 8 to 10 carbon atoms, preferably a saturated alkyl group, more preferably a saturated, linear, alkyl group; and Y is an alkyl, alkenyl, hydroxy or carboxy substituted alkyl or alkenyl, having from about 2 to about 20 carbon atoms, preferably from about 3 to about 14 carbon atoms.

It has been found that this select group of synthetic esters provides improved wet hair feel when used in combination with the essential components of the shampoo composition herein, and in particular when used in combination with the cationic hair conditioning polymer of the shampoo composition. These synthetic esters improve wet hair feel by reducing the slimy or excessively conditioned feel of wet hair that has been conditioned by a cationic hair conditioning polymer. By application of the shampoo composition herein, cleansed and conditioned hair remains detangled and silky during and after the shampooing process, but the excessively conditioned or slimy wet hair feel undesirably associated with good conditioning performance is minimized or eliminated.

Specific non limiting examples of suitable synthetic esters for use in the shampoo composition include P-43 (C8-C10 triester of trimethylolpropane), MCP-684 (tetraester of 3,3 diethanol-1,5 pentadiol), MCP 121 (C8-10 diester of adipic acid), all of which are available from Mobil Chemical Company, Edison, N.J., U.S.A.

### C. Hair Conditioning Oily Liquid

The shampoo compositions of the present invention may comprise a nonvolatile, water insoluble, organic, oily liquid as a hair conditioning agent. The hair conditioning oily liquid can add shine and luster to the hair. Additionally, it can also enhance dry combing and dry hair feel. The hair conditioning oily liquid is typically present in the compositions at a level of from about 0.05% to about 5%, by weight of the composition, preferably from about 0.2% to about 3%, more preferably from about 0.5% to about 1%.

Herein, “nonvolatile” means the oily material exhibits very low or no significant vapor pressure at ambient conditions (e.g., 1 atmosphere, 25° C.), as is understood in the art. The nonvolatile oily materials preferably have a boiling point at ambient pressure of about 250° C. or higher.

Herein, “water insoluble” means the oily liquid is not soluble in water (distilled or equivalent) at a concentration of 0.1%, at 25° C.

The hair conditioning oily liquids hereof generally will have a viscosity of about 3 million cs or less, preferably about 2 million cs or less, more preferably about 1.5 million cs or less.

The hair conditioning oily materials hereof are liquids selected from the group consisting of hydrocarbon oils and fatty esters. The fatty esters hereof are characterized by having at least 10 carbon atoms, and include esters with hydrocarbyl chains derived from fatty acids or alcohols, e.g., mono-esters, polyhydric alcohol esters, and di- and tri-carboxylic acid esters. The hydrocarbyl radicals of the fatty esters hereof can also include or have covalently bonded thereto other compatible functionalities, such as amides and alkoxy moieties (e.g., ethoxy or ether linkages, etc.).

Hydrocarbon oils include cyclic hydrocarbons, straight chain aliphatic hydrocarbons (saturated or unsaturated), and branched chain aliphatic hydrocarbons (saturated or unsaturated). Straight chain hydrocarbon oils will preferably contain from about 12 to about 19 carbon atoms, although it is not necessarily meant to be limit the hydrocarbons to this range. Branched chain hydrocarbon oils can and typically may contain higher numbers of carbon atoms. Also encompassed herein are polymeric hydrocarbons of alkenyl monomers, such as C₂-C₆ alkenyl monomers. These polymers can be straight or branched chain polymers. The straight chain polymers will typically be relatively short in length, having a total number of carbon atoms as described above for straight chain hydrocarbons in general. The branched chain polymers can have substantially higher chain length. The number average molecular weight of such materials can vary widely, but will typically be up to about 500, preferably from about 200 to about 400, more preferably from about 300 to about 350. Specific examples of suitable materials include paraffin oil, mineral oil, saturated and unsaturated dodecane, saturated and unsaturated tridecane, saturated and unsaturated tetradecane, saturated and unsaturated pentadecane, saturated and unsaturated hexadecane, and mixtures thereof. Branched-chain isomers of these compounds, as well as of higher chain length hydrocarbons, can also be used. Exemplary branched-chain isomers are highly branched saturated or unsaturated alkanes, such as the permethyl-substituted isomers, e.g., the permethyl-substituted isomers of hexadecane and eicosane, such as 2,2,4,4,6,6,8,8-dimethyl-10-methylundecane and 2,2,4,4,6,6-dimethyl-8-methylnonane, sold by Permethyl Corporation. A preferred hydrocarbon polymer is polybutene, such as the copolymer of isobutylene and butene. A commercially available material of this type is L-14 polybutene from Amoco Chemical Co. (Chicago, Ill., U.S.A.).

Monocarboxylic acid esters hereof inlude esters of alcohols and/or acids of the formula R′COOR wherein alkyl or alkenyl radicals and the sum of carbon atoms in R′ and R is at least 10, preferably at least 20.

Fatty esters useful herein include, for example, alkyl and alkenyl esters of fatty acids having aliphatic chains with from about 10 to about 22 carbon atoms, and alkyl and alkenyl fatty alcohol carboxylic acid esters having an alkyl and/or alkenyl alcohol-derived aliphatic chain with about 10 to about 22 carbon atoms, and combinations thereof. Examples include isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

The mono-carboxylic acid ester however need not necessarily contain at least one chain with at least 10 carbon atoms, so long as the total number of aliphatic chain carbon atoms is at least 10. Examples include diisopropyl adipate, diisohexyl adipate, and diisopropyl sebacate.

Di- and tri-alkyl and alkenyl esters of carboxylic acids can also be used. These include, for example, esters of C₄-C₈ dicarboxylic acids such as C₁-C₂₂ esters (preferably C₁-C₆) of succinic acid, glutaric acid, adipic acid, hexanoic acid, heptanoic acid, and octanoic acid. Specific examples include isocetyl stearyol stearate, diisopropyl adipate, and tristearyl citrate.

Polyhydric alcohol esters include alkylene glycol esters, for example ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters for use herein.

Glycerides include mono-, di-, and tri-glycerides. More specifically, included are the mono-, di-, and tri-esters of glycerol and long chain carboxylic acids, such as C₁₀-C₂₂ carboxylic acids. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include triolein and tristearin glyceryl dilaurate. Preferred glycerides are di-, and tri-glycerides. Especially preferred are triglycerides.

### IV. Anti-Dandruff Agent

The anti-dandruff and conditioning shampoo compositions of the present invention comprise from about 0.1% to about 4%, by weight of the composition, preferably from about 0.1% to about 3%, most preferably from about 0.3% to about 2%, of an anti-dandruff agent suitable for application to the hair or skin. The anti-dandruff agent provides the shampoo compositions with anti-microbial activity. The anti-dandruff agent may be particulate or soluble. Suitable, non-limiting examples of particulate anti-dandruff agents include: pyridinethione salts, selenium sulfide, particulate sulfur, and mixtures thereof. Preferred are pyridinethione salts. A suitable, non-limiting example of a soluble anti-dandruff agents is ketoconazole. Such anti-dandruff agent should be physically and chemically compatible with the essential components of the composition, and should not otherwise unduly impair product stability, aesthetics or performance.

1. Pyridinethione salts

Pyridinethione anti-dandruff particulates, especially 1-hydroxy-2-pyridinethione salts, are highly preferred particulate anti-dandruff agents for use in the anti-dandruff and conditioning shampoo compositions of the present invention. The concentration of pyridinethione anti-dandruff particulate typically ranges from about 0.1% to about 4%, by weight of the composition, preferably from about 0.1% to about 3%, most preferably from about 0.3% to about 2%. Preferred pyridinethione salts include those formed from heavy metals such as zinc, tin, cadmium, magnesium, aluminum and zirconium, preferably zinc, more preferably the zinc salt of 1-hydroxy-2-pyridinethione (known as “zinc pyridinethione” or “ZPT”), most preferably 1-hydroxy-2-pyridinethione salts in platelet particle form, wherein the particles have an average size of up to about 20μ, preferably up to about 5μ, most preferably up to about 2.5μ. Salts formed from other cations, such as sodium, may also be suitable. Pyridinethione anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982, all of which are incorporated herein by reference. It is contemplated that when ZPT is used as the anti-dandruff particulate in the shampoo compositions herein, that the growth or re-growth of hair may be stimulated or regulated, or both, or that hair loss may be reduced or inhibited, or that hair may appear thicker or fuller.

2. Selenium sulfide

Selenium sulfide is a particulate anti-dandruff agent suitable for use in the anti-dandruff and conditioning shampoo compositions of the present invention, effective concentrations of which range from about 0.1% to about 4%, by weight of the composition, preferably from about 0.3% to about 2.5%, more preferably from about 0.5% to about 1.5%. Selenium sulfide is generally regarded as a compound having one mole of selenium and two moles of sulfur, although it may also be a cyclic structure that conforms to the general formula SeₓS_{y}, wherein x+y=8. Average particle diameters for the selenium sulfide are typically less than 15 μm, as measured by forward laser light scattering device (e.g. Malvern 3600 instrument), preferably less than 10 μm. Selenium sulfide compounds are described, for example, in U.S. Pat. No. 2,694,668; U.S. Pat. No. 3,152,046; U.S. Pat. No. 4,089,945; and U.S. Pat. No. 4,885,107, all of which descriptions are incorporated herein by reference.

3. Sulfur

Sulfur may also be used as the particulate anti-dandruff agent in the anti-dandruff and conditioning shampoo compositions of the present invention. Effective concentrations of the particulate sulfur are typically from about 1% to about 4%, by weight of the composition, preferably from about 2% to about 4%.

4. Ketoconazole

Ketoconazole may also be used as the soluble anti-dandruff agent in the anti-dandruff and conditioning shampoo compositions of the present invention. Effective concentrations of ketoconazole are typically from about 0.1% to about 4%, by weight of the composition, preferably from about 0.3% to about 2%.

### V. Aqueous Carrier

The shampoo compositions of the present invention are typically liquids which, preferably, are pourable at room temperature. The compositions hereof preferably comprise an aqueous carrier, i.e., water, which will generally be present at a level of about 20% to about 95% by weight of the composition, preferably from about 50 to about 94%, more preferably from about 60% to about 85% by weight, for pourable, liquid formulations.

### VI. Optional Components

The present compositions may also comprise a variety of non-essential, optional shampoo components suitable for rendering such compositions more cosmetically or aesthetically acceptable or to provide them with additional usage benefits, provided that the optional components are physically and chemically compatible with the essential component described herein, or do not otherwise unduly impair product stability, aesthetics or performance. A variety of such ingredients are well-known to those skilled in the art, and these include without limiting the invention thereto: pearlescent aids, such as coated mica, ethylene glycol distearate; opacifiers, such as TiO₂; preservatives, such as benzyl alcohol, 1,3-bis(hydroxymethyl)-5,5-dimethyl-2,3-imidazolidinedione (e.g., GlydantR, Glyco, Inc., Greenwich, Conn., USA), methylchloroisothiazolinone (e.g., KathonR, Rohm & Haas Co., Philadelphia, Pa., USA), methyl paraben, propyl paraben, and imidazolidinyl urea; fatty alcohols, such as cetearyl alcohol, cetyl alcohol, and stearyl alcohol; sodium chloride; ammonium chloride; sodium sulfate; ethyl alcohol; pH adjusting aids, such as citric acid, sodium citrate, succinic acid, phosphoric acid, monosodium phosphate, disodium phosphate, sodium hydroxide, and sodium carbonate; coloring agents or dyes; perfumes; and sequestering agents, such as disodium ethylenediamine tetra-acetate, organic solvents or diluents, foam boosters, additional surfactants or cosurfactants (nonionic, cationic, zwitterionic), pediculocides, preservatives, proteins, skin active agents, suspending agents, styling polymer, sunscreens, thickeners, vitamins and viscosity adjusting agents.

Another optional ingredient that can be advantageously used is an anti-static agent. The anti-static agent should not unduly interfere with the in-use performance and end-benefits of the shampoo; particularly, the anti-static agent should not interfere with the anionic detersive surfactant. Suitable anti-static agents include, for example, tricetyl methyl ammonium chloride.

Typically, from about 0.1% to about 5% of such anti-static agent is incorporated into the shampoo compositions.

The compositions of the present invention can also be in other forms, such as gels, mousses, etc. In such cases, appropriate components known in the art such as gelling agents (e.g., hydroxyethyl cellulose), etc. can be included in the compositions. Gels will typically contain from about 20% to about 90% water. Mousses will be a low viscosity composition and will be packaged as a sprayable liquid according to techniques well known in the art, typically in an aerosol cannister including a propellant or a means for generating an aerosol spray.

In an embodiment of the present invention a suspending agent may be present for the insoluble hair conditioning agent. Suitable suspending agents are long chain acyl derivatives, long chain amine oxides, and mixtures thereof, wherein such suspending agents are present in the shampoo compositions in crystalline form. A variety of such suspending agents are described in U.S. Pat. No. 4,741,855, Grote et al., issued May 3, 1988. Especially preferred is ethylene glycol distearate.

Also included among the long chain acyl derivatives useful as suspending agents are the N,N-di(hydrogenated) C₈-C₂₂ (preferably C₁₂-C₂₂, more preferably C₁₆-C₁₈) amido benzoic acid, or soluble salt (e.g., K, Na salts) thereof particularly N,N-di(hydrogenated)-tallow amido benzoic acid which is commercially marketed by Stepan Company (Northfield, Ill., USA).

Another useful suspending agent for the silicone conditioning agents of the present compositions is xanthan gum as described in U.S. Pat. No. 4,788,006, Bolich et al., issued Jun. 5, 1984. The combination of long chain acyl derivatives and xanthan gum as a suspending system for silicone is described in U.S. Pat. No. 4,704,272, Oh et al., issued Nov. 3, 1987, and may also be used in the present compostions.

Generally, the shampoo compositions will comprise from about 0.1% to about 5.0%, preferably from about 0.5% to about 3.0%, of the suspending agent to suspend the silicone conditioning agent. The suspending agent may have additional benefits such as enhancing the coacervate formation and could thus be used in the absence of an insoluble hair conditioning agent.

Though the suspending agent component may act to thicken the present compositions to some degree, the present compositions may also optionally contain other thickeners and viscosity modifiers such as an ethanolamide of a long chain fatty acid (e.g., polyethylene (3) glycol lauramide and coconut monoethanolamide) and ammonium xylene sulfonate.

These optional components generally are used individually in the compositions of the present invention at a level of from about 0.01% to about 10%, preferably from about 0.05% to about 5.0% of the shampoo composition. This list of optional components is not meant to be exclusive, and other optional components can be used.

### VII. Method of Manufacture

The shampoo compositions of the present invention can be prepared by using various formulation and mixing techniques or methods known in the art for preparing surfactant or conditioning compositions, or other similar compositions.

### VIII. Method of Use

The shampoo compositions of the present invention are utilized conventionally, i.e., the hair is shampooed by applying an effective amount of the shampoo composition to the scalp, and then rinsing it out with water. Application of the shampoo to the scalp in general, encompasses massaging or working the shampoo in the hair such that all or most of the hair on the scalp is contacted. herein, “effective amount” means an amount which is effective in cleaning and conditioning the hair. Generally, from about 1 g to about 50 g, preferably from about 1 g to about 20 g, of the composition is applied for cleaning and conditioning the hair. Preferably, the shampoo is applied to hair in a wet or damp state.

This method for cleansing and conditioning the hair comprises the steps of: a) wetting the hair with water, b) applying an effective amount of the shampoo composition to the hair, and c) rinsing the shampoo composition from the hair using water. These steps can be repeated as many times as desired to achieve the desired cleansing and conditioning benefit.

The compositions hereof can also be useful for cleaning and conditioning the skin. For such applications, the composition would be applied to the skin in a conventional manner, such as by rubbing or massaging the skin with the composition, optionally in the presence of water, and then rinsing it away with water.

### IX. System Use

The shampoo composition of the present invention is preferably used together with a conditioner composition as a system, i.e., after rinsing the shampoo composition, conditioner compositions are preferably applied to the hair for obtaining further conditioning benefits.

The conditioner compositions useful herein preferably comprise monoalkyl trimethyl ammonium salts and fatty alcohols. It is believed that; the monoalkyl trimethyl ammonium salts, together with the fatty alcohols, provide a gel matrix suitable for providing various conditioning benefits such as softness, moisturized feel, and fly-away control on dry hair. The gel matrix may become unstable or, at worst, become destroyed in the presence of certain components. Such components include high levels of anionic surfactants and polymers having anionic moieties. A highly preferred composition is substantially free of such components.

The monoalkyl trimethyl ammonium salts useful in the present invention have the formula:

[chemical structure]

wherein R₁ is selected from an aliphatic group of from 12 to 30 carbon atoms, preferably from 16 to 22 carbon atoms, more preferably 22 carbon atoms; and X is a salt-forming anion selected from halogen (e.g. chloride, bromide), acetate, citrate, lactate, glycolate, phosphate, nitrate, sulfonate, sulfate, alkylsulfate, and alkyl sulfonate radicals, preferably selected from halogen such as chloride, and alkylsulfate such as methosulfate. The aliphatic groups may contain, in addition to carbon and hydrogen atoms, ether linkages as well as amido groups among other groups. It is believed that; monoalkyl trimethyl ammonium salts deposit more effectively on the hair compared to other cationic conditioning agents such as cationic polymers, other cationic surfactants such as those comprising alkyl ammonium salts having 2 or more long alkyl groups, and those comprising tertiary amines such as amidoamines and acids. It is also believed that; the improved deposition of monoalkyl trimethyl ammonium salts compared to other cationic conditioning agents can be obtained, when the monoalkyl trimethyl ammonium salts are contained in conditioner compositions used as a system together with the shampoo compositions of the present invention, especially used as a system together with the shampoo composition containing betaine surfactants. Among the monoalkyl trimethyl ammonium salts of general formula (I), nonlimiting examples of preferred monoalkyl trimethyl ammonium salts include: behenyl trimethyl ammonium chloride available, for example, with tradename INCROQUAT TMC-80 from Croda and ECONOL TM22 from Sanyo Kasei; cetyl trimethyl ammonium chloride available, for example, with tradename CA-2350 from Nikko Chemicals; steary trimethyl ammonium chloride available, for example, with tradename Varisoft TS50 and Varisoft TSC from Witco; and hydrogenated tallow alkyl trimethyl ammonium. More preferred is behenyl trimethyl ammonium chloride. The monoalkyl trimethyl ammonium salts are included in the conditioner composition at a level of from about 0.1% to about 5%, preferably from about 0.5% to about 4%, and more preferably from about 1% to about 3%, still more preferably from about 1.5% to about 2.5% by weight of the total composition.

The fatty alcohol useful herein has an alkyl group having preferably from 12 to 30 carbon atoms, more preferably from 16 to 22 carbon atoms, still preferably from 16 to 18 carbon atoms. Preferably, the fatty alcohol useful herein has a melting point higher than 30° C. Examples of such fatty alcohol materials include stearyl-, cetyl-, myristyl-, behenyl-, and lauryl alcohols, and mixtures thereof. Highly preferred of the fatty alcohols are cetyl and stearyl alcohol or mixtures thereof. Commercially available fatty alcohols useful herein include: cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan). The fatty alcohols are included in the conditioner composition at a level of from about 1% to about 15%, preferably from about 2% to about 14%, and more preferably from about 3.5% to about 8.5%, still more preferably from about 5% to about 7% by weight of the total composition.

The hair conditioner compositions also comprise water. It is generally present at a level of from about 20% to about 98.9%, preferably from about 60% to about 95%, more preferably from about 80% to about 90% by weight of the total composition.

The hair conditioner compositions may also contain other materials which provide conditioning benefits. Such materials useful herein include, for example, silicone compounds, polypropylene glycols, polyethylene glycols, other oils such as pentaerythritol tetraisostearate than fatty alcohols described above, cationic conditioning agents such as cationic polymers and cationic surfactants other than monoalkyl trimethyl ammonium salts described above. If included, such materials are included in the conditioner compositions at a level of from about 0.01% to about 10%, preferably no more than 5% by weight of the total composition.

The hair conditioner composition may also include a variety of other components suitable for rendering such compositions acceptable for use. Such components are generally well known to those skilled in the art and may include for examples preservatives such as benzyl alcohol, trimethyl paraben, propyl paraben and imidazolidinyl urea, thickeners and viscosity modifiers such as a hydroxy ethyl cellulose and xanthan gum, pH adjusting agents such as citric acid, sodium citrate, succinic acid, phosphoric acid, sodium hydroxide, sodium carbonate etc; perfume, dyes and sequestering agents such as disodium ethylene diamine tetraacetate. Such agents generally are used individually at a level of from about 0.01% to about 10%, preferably from about 0.1% to about 5% by weight of the total composition.

One preferred embodiment of the hair conditioner compositions is shown in table below.

Hair conditioner composition

| Description | wt % |
|---|---|
| Water | Q.S. to 100 |
| Cetyl Alcohol | 2.25 |
| Stearyl Alcohol | 4.05 |
| Behenyl trimethyl ammonium chloride | 1.8 |
| Benzyl Alcohol | 0.4 |
| Kathon CG | 0.033 |
| Disodium EDTA | 0.127 |
| Perfume | 0.5 |
| dl-Panthenyl ethyl ether | 0.05 |
| dl-Panthenol | 0.05 |

The hair conditioner composition may be prepared by following method: Monoalkyl trimethyl ammonium salts and fatty alcohols are added to hot (70-80° C.) water. Then, the mixture is slowly cooled down to 45-55° C. where other ingredients including fatty alcohols are added, followed by cooling to room temperature.

### EXAMPLES

The following examples illustrate specific embodiments of the shampoo composition of the present invention, but are not intended to be limiting thereof. It will be appreciated that other modifications of the present invention within the skill of those in the hair care formulation art can be undertaken without departing from the spirit and scope of this invention. These exemplified embodiments of the shampoo compositions of the present invention provide cleansing of hair and improved hair conditioning performance.

All parts, percentages, and ratios herein are by weight unless otherwise specified. Some components may come from suppliers as dilute solutions. The levels given reflect the weight percent of the active material, unless otherwise specified. The excluded diluents and other materials are included in as “Minors”.

### EXAMPLES I, II and III

The following is a shampoo composition of the present invention:

| Supplier name/Description | 1 | 2 | 3 |
|---|---|---|---|
| Water-USP Purified & Minors | Q.S. to 100 | Q.S. to 100 | Q.S. to 100 |
| Ammonium Laureth Sulfate | 10.0000 | 12.5000 | 12.0000 |
| Ammonium Lauryl Sulfate | 6.0000 | 1.5000 | 2.0000 |
| Cocamidopropyl Betaine | | 2.7000 | |
| Sodium Lauroamphoacetate | | | 2.0000 |
| Cocamide MEA | 0.8000 | 0.8000 | 0.8000 |
| Cetyl Alcohol | 0.9000 | 0.6000 | 0.6000 |
| Ethylene Glycol Distearate | 1.5000 | 1.5000 | |
| Dimethicone Viscasil 330,000 | 1.3500 | | |
| Dow Corning 1664 | | 1.0000 | |
| 300 nm/60M emulsion | | | |
| Polyquaternium-10 (LR30M) | 0.5000 | 0.1500 | 0.5000 |
| Polyox PEG7M | 0.1000 | | |
| Puresyn 6 (1-decene | 0.3000 | | |
| homopolymer) | | | |
| Perfume | 0.5000 | 0.5000 | 0.5000 |
| Citric Acid | 0.0400 | 0.0400 | 0.4000 |
| Sodium Citrate Dihydrate | 0.3972 | 0.3972 | 0.3972 |
| Disodium EDTA | 0.0993 | 0.0993 | 0.0993 |
| Kathon | 0.0005 | 0.0005 | 0.0005 |
| Sodium Benzoate | 0.2500 | 0.2500 | 0.2500 |
| Sodium Chloride | 0-3 | 0-3 | 0-3 |
| Ammonium Xylene Sulfonate | 0-3 | 0-3 | 0-3 |

The compositions illustrated in the three examples were prepared in the following manner (all percentages are based on weight unless otherwise specified).

For each of the compositions, 36% of ammonium laureth sulfate (solution basis, 25% active) and 9.75% water was added to a jacketed mix tank and heated to about 74° C. with slow agitation to form a surfactant solution. Then, where present, Citric Acid, Sodium Citrate, Sodium Benzoate, Disodium EDTA, Cocamide MEA Polyox, Polyquaternium-10, Puresyn 6, and Cetyl alcohol, were added to the tank and allowed to disperse. Ethylene glycol distearate (EGDS) was then added, with the exception of Example III, to the mixing vessel, and melted. After the EGDS was well dispersed (after about 10 minutes) Kathon was added and mixed into the surfactant solution. This mixture was passed through a heat exchanger where it was cooled to about 35° C. and collected in a finishing tank. As a result of this cooling step, the ethylene glycol distearate crystallized to form a crystalline network in the product. The remainder of the ammonium laureth sulfate, lauryl sulfate, sodium lauroamphoacetate, Cocamidopropyl Betaine, Perfume and remaining water were added to the finishing tank with ample agitation to insure a homogeneous mixture. Sodium Chloride or Ammonium Xylene Sulfonate were added as needed to adjust viscosity to the desired range, and Dimethicone (premixed as follows) or Dow Coming 1664 were added and mixed with ample agitation to insure a homogeneous mixture. The dimethicone premix was prepared by adding 70% dimethicone, 30% ammonium laureth-3 sulfate (solution basis, 25% active), all by weight of the dimethicone premix, to a high shear mixing vessel and mixed for about 30 minutes to a silicone particle size of ˜27 microns.

It is understood that the examples and embodiments described herein are for illustrative purposes only and that various modifications or changes in light thereof will be suggested to one skilled in the art without departing from the scope of the invention.

What is claimed is:

1. A hair conditioning shampoo composition comprising:
(a) from about 5% to about 50%, by weight, of a surfactant component selected from the group consisting of anionic surfactants, amphoteric surfactants, or a combination of anionic and amphoteric or zwitterionic surfactants where the amphoteric surfactants are anionic or zwitterionic at the pH of the composition;
(b) from about 0.01% to about 5%, by weight, of a water soluble, organic, cationic polymer hair conditioning agent having a cationic charge density of from about 0.1 meq/gram to about 1.2 meq/gram and wherein the water soluble, organic, cationic polymer hair conditioning agent has a molecular weight greater than 600,000; and
(c) an aqueous carrier;

2. The shampoo composition of claim 1, wherein the cationic polymer is in a complex coacervate phase in the shampoo composition or forms a complex coacervate upon dilution of the shampoo composition with water.

3. The shampoo composition of claim 2, wherein the cationic polymer exists in a complex coacervate which forms upon dilution of the shampoo composition with water at a water:shampoo composition weight ratio of about 20:1.

4. The shampoo composition of claim 1, wherein said cationic charge density is from about 0.1 meq/gram to about 1.2 meq/gram.

5. The shampoo composition of claim 4, wherein the cationic charge density is from about 0.3 meq/gram to about 0.8 meq/gram.

6. The shampoo composition of claim 1, wherein the water soluble, organic, cationic polymer hair conditioning agent is a cationic cellulose polymer hair conditioning agent.

7. The shampoo composition of claim 6, wherein the cationic cellulose polymer hair conditioning agent is Polyquaternium-10 sold under the tradename Polymer LR-30M.

8. The shampoo composition of claim 1, wherein the water soluble, organic, cationic polymer hair conditioning agent has a molecular weight from about 800,000 to about 2 million.

9. The shampoo composition of claim 8, wherein the water soluble, organic, cationic polymer hair conditioning agent has a molecular weight from about 1 million to about 1.5 million.

10. The shampoo composition of claim 1, wherein the shampoo composition further comprises an insoluble hair conditioning agent.

11. The shampoo composition of claim 10, wherein the insoluble conditioning agent is a dispersed, insoluble, nonvolatile, nonionic silicone hair conditioning agent.

12. The shampoo composition of claim 1, further comprising a suspending agent.

13. The shampoo composition of claim 12, wherein the silicone hair conditioning agent comprises a mixture of polydimethylsiloxane gum and polydimethylsiloxane fluid.

14. The shampoo composition of claim 12, wherein the silicone hair conditioning agent comprises polydimethylsiloxane.

15. The shampoo composition of claim 10, comprising from about 0.005% to about 5% of the insoluble hair conditioning agent.

16. The shampoo composition of claim 10, wherein the insoluble hair conditioning agent is selected from the group consisting of hydrocarbon oils, fatty esters having at least 10 carbon atoms, synthetic esters, silicones, synthetic esters and mixtures thereof.

17. The shampoo composition of claim 16, wherein the fatty esters are selected from the group consisting of alkyl and alkenyl esters of fatty acids, alkyl and alkenyl esters of fatty alcohols, polyhydric alcohol esters, dicarboxylic acid esters, tricarboxylic acid esters, and mono-, di-, and tri-glycerides, and mixtures thereof.

18. The shampoo composition of claim 1, wherein the cationic polymer hair conditioning agent is selected from the group consisting of cationic cellulose, cationic starch, cationic guar, dialkylaminoalkyl acrylate, dialkylaminoalkyl methacrylate, monoalkylaminoalkyl acrylate, monoalkylaminoalkyl metharcylate, trialkyl methacryloxyalkyl ammonium salt, trialkyl acryloxyalkyl ammonium salt, diallyl quaternary ammonium salts, pyridinium, imidazolium, quaternized pyrrolidone and mixtures thereof.

19. The shampoo composition of claim 18, wherein the cationic polymer hair conditioning agent is selected from the group consisting of cationic cellulose, cationic starch, cationic guar, copolymers of 1-vinyl-2-pyrrolidone and 1-vinyl-3-methyl imidazolium salt, copolymers of acrylamide and dimethyl diallylammonium salt, copolymers of 1-vinyl-2-pyrrolidone and dimethylaminoethyl methacrylate, and mixtures thereof.

20. The shampoo composition of claim 19, wherein the cationic polymer is selected from the group consisting of cationic cellulose, cationic starch, cationic guar and mixtures thereof.

21. The shampoo composition of claim 1, wherein the cationic polymer conditioning agent is a cationic polysaccharide polymer having a cationic charge density of from about 0.1 meq/gram to about 1.2 meq/gram and wherein the cationic polysaccharide has a molecular weight greater than about 600,000.

22. The shampoo composition of claim 21 further comprising an insoluble hair conditioning agent.

23. A hair conditioning shampoo composition comprising:
(a) from about 5% to about 50%, by weight, of a surfactant component selected from the group consisting of anionic surfactants, amphoteric surfactants, or a combination of anionic and amphoteric or zwitterionic surfactants where the amphoteric surfactants are anionic or zwitterionic at the pH of the composition;
(b) from about 0.01% to about 5%, by weight, of a water soluble, organic, cationic cellulose polymer hair conditioning agent having a cationic charge density of from about 0.3 meq/gram to about 0.8 meq/gram and wherein the water soluble, organic, cationic cellulose polymer hair conditioning agent has a molecular weight from about 800,000 to about 2 million;
(c) an insoluble hair conditioning agent wherein the insoluble hair conditioning agent is silicone;
(d) a suspending agent; and
(e) an aqueous carrier.

24. The shampoo composition of claim 1 wherein the surfactant is selected from the group consisting of a combination of anionic and amphoteric surfactants, a combination of anionic and zwitterionic surfactants, and mixtures thereof.

25. The shampoo composition of claim 12 wherein the suspending agent is ethylene glycol distearate.

26. The shampoo composition of claim 1, which further comprises an anti-dandruff agent.

27. The shampoo composition of claim 21, wherein the anti-dandruff agent is selected from the group consisting of pyridinethione salts, selenium sulfide, particulate sulfur, ketoconazole and mixtures thereof.

28. A method for shampooing hair, the method comprising applying to hair an effective amount of the shampoo composition of claim 1 for cleaning and conditioning the hair and then rinsing the shampoo composition from the hair.

29. A method for shampooing hair, the method comprising applying to hair an effective amount of the shampoo composition of claim 10 for cleaning and conditioning the hair and then rinsing the composition from the hair.

30. A method for shampooing hair, the method comprising applying to hair an effective amount of the shampoo composition of claim 21 for cleaning and conditioning the hair and then rinsing the composition from the hair.

31. A method for shampooing hair, the method comprising applying to hair an effective amount of the shampoo composition of claim 22 for cleaning and conditioning the hair and then rinsing the composition from the hair.

32. A system use of the shampoo composition according to claim 1 and a conditioner composition wherein the conditioner composition comprises:
(a) from about 0.1% to about 5.0% by weight, of a monoalkyl trimethyl ammonium salt; and
(b) from about 1% to about 15%, by weight, of a fatty alcohol.

33. The system use according to claim 32 wherein the monoalkyl trimethyl ammonium salt is a monoalkyl trimethyl ammonium chloride having an alkyl group of from 12 to 30 carbon atoms.

34. The system use according to claim 33 wherein the monoalkyl trimethyl ammonium chloride is behenyl trimethyl ammonium chloride.

35. The system use according to claim 32 wherein the fatty alcohol has an alkyl group having from 12 to 30 carbon atoms.

36. The system use according to claim 35 wherein the fatty alcohol has a melting point of higher than 30° C.

37. The system use according to claim 36 wherein the fatty alcohol is selected from the group consisting of cetyl alcohol, stearyl alcohol, and mixtures thereof.
